(19) United States
(12) Patent Application Publication
(10) Pub. No.: US 2014/0328896 A1
(43) Pub. Date: Nov. 6, 2014

(54) COMPOSITIONS AND METHODS FOR MODIFYING GASTROINTESTINAL FLORA

(71) Applicants: Dennis Jewll, Lawrence, KS (US); Luis J. Montelongo, Lawrence, KS (US); Sarah Martinez, Lawrence, KS (US); Lynda Melendez, Topeka, KS (US)

(72) Inventors: Dennis Jewll, Lawrence, KS (US); Luis J. Montelongo, Lawrence, KS (US); Sarah Martinez, Lawrence, KS (US); Lynda Melendez, Topeka, KS (US)

(73) Assignee: HILL'S PET NUTRITION, INC., Topeka, KS (US)

(21) Appl. No.: 14/368,310

(22) PCT Filed: Dec. 29, 2011
(86) PCT No.: PCT/US2011/067713

§ 371 Date: Jun. 24, 2014

(51) Int. Cl. A61K 31/19; A23K 1/17; A23K 1/18; A23L 1/30; A61K 45/06; A23K 1/16

(52) U.S. Cl. CPC A61K 31/19; A61K 45/06; A23K 1/1646; A23K 1/1846; A23K 1/1853; A23K 1/1866; A23L 1/3014; A23K 1/17; A23K 1/1643; A23V 2002/00
USPC 424/442; 514/557; 514/200; 514/37; 514/198; 514/253.08; 514/422; 514/39; 514/398; 514/2.9; 514/327; 424/94.6; 424/93.1; 424/725; 424/747; 424/756; 426/2; 426/61; 426/71

(57) ABSTRACT

Provided are methods and compositions for enhancing the gastrointestinal health of an animal in need thereof by administering a gastrointestinal health maintaining or improving amount of a composition comprising an effective amount of lactic acid. The administered composition may also comprise one or more prebiotic, probiotic, symbiotic, anti bacterial, or anti-diarrhea agents.

### FIELD

The present invention relates to compositions and methods for modifying the bacterial microflora of the gastrointestinal tract in animals in need thereof. The disclosed methods also relate to enhancing and improving the gastrointestinal health of an animal afflicted with a condition arising from, associated with, or characterized by poor gastrointestinal health. The disclosed methods comprise administering a composition comprising an effective amount of lactic acid. Also disclosed are compositions comprising lactic acid that are useful in the described methods.

### BACKGROUND

Poor gastrointestinal health is not uncommon and is associated with, contributes to, exacerbates, or causes any number of conditions affecting the overall health and well-being of animals, particularly mammals. Conditions related to poor gastrointestinal health can be quite serious and require medical attention. They include, for example, Crohn's disease and irritable bowel disease, as well as other similar, chronic conditions. Other conditions related to poor gastrointestinal health that are less serious and can be essentially self-limiting include, for example, food-borne viruses and intestinal flu that often result in diarrhea, poor stool quality, or other symptoms of poor gastrointestinal health. Poor gastrointestinal health results from various causes. For example, intestinal bacterial overgrowth (IBO) occurs in people and companion animals such as dogs, cats, and horses. IBO may be caused by poor motility, food retention, or decreased gastric acidity. Further, animals must efficiently and properly digest food and absorb these dietary nutrients in order to maintain good health. However, poor gastrointestinal health can interfere with the ordinary digestion of food and adversely affect an animal's health and well-being.

Many gastrointestinal disorders are accompanied by diarrhea, a loose watery stool that can be extremely unpleasant to an animal and its caregiver. Further, some foods tend to create or exacerbate diarrhea. For example, U.S. Pat. No. 6,280,779 discloses that diarrhea can be a significant problem with pets consuming a “chunks and gravy” diet. Similarly, poor stool quality is often indicative of milder forms of poor gastrointestinal health that do not result in diarrhea.

Current methods for maintaining and improving gastrointestinal health often involve modifying the diet, administering various food ingredients thought to affect gastrointestinal health, or administering various drugs thought to be useful for maintaining or improving gastrointestinal health. These methods, while useful, have not solved the problem. There is, therefore, a need for new methods and compositions for maintaining and improving, and, more specifically, for enhancing gastrointestinal health in an animal.

### BRIEF SUMMARY

The present invention is based, in part, on the discovery that administration of a composition comprising lactic acid can enhance the gastrointestinal health of a mammal in need thereof. In one aspect of this embodiment of the present invention, the method comprises administration of a composition comprising an effective amount of lactic acid to an animal in need of enhancement of gastrointestinal health.

In one embodiment of the methods disclosed herein, the composition comprising lactic acid is administered to an animal diagnosed as suffering from or diagnosed as susceptible to a condition related to poor gastrointestinal health. In particular aspects of this embodiment, the condition comprises diarrhea, irritable bowel disease, nutrient malabsorption, or a combination of two or more thereof.

In certain embodiments, the animal in need of enhancement of gastrointestinal health is a companion animal, e.g., the animal is a canine or feline.

In other embodiments of the disclosed invention, the administered composition also comprises one or more ingredients selected from among probiotics, prebiotics, synbiotics, microbial exopolysaccharides, and combinations thereof.

In another embodiment of the disclosed methods, the administered composition is a nutritionally complete pet food composition. In a particular aspect of this embodiment, the pet food composition is a dry pet food composition. In another aspect of this embodiment, the dry pet food composition comprises a matrix of ingredients or particles that comprise a coating layer, wherein the coating layer comprises lactic acid.

In another embodiment of the disclosed methods, the administered composition is a nutritionally complete pet food composition. In a particular aspect of this embodiment, the pet food composition is a wet pet food composition.

In another embodiment, the administered composition is a food supplement. In other embodiments, the composition comprising lactic acid is prepared in the form of a treat, a snack, or an at least partially edible chew or toy.

In another aspect of the methods disclosed herein, the administered composition comprising lactic acid enhances the gastrointestinal health of a mammal in need thereof by decreasing the gastrointestinal level of at least one genus or species of deleterious bacteria. In one aspect of this embodiment, the deleterious gastrointestinal level of bacteria of the genus Enterococcus is reduced. In another aspect of this embodiment, the deleterious gastrointestinal level of bacteria of the genus Desulfovibrio is reduced. In another aspect of this embodiment, the deleterious gastrointestinal level of bacteria of the genus Helicobacter is reduced. In a further aspect of this embodiment, deleterious gastrointestinal levels of one or more of the following species of bacteria are reduced: Salmonella typhimurium, Staphylococcus aureus, Clostridium perfringens, Clostridium difficile, Vibrio parahaemolyticus, Vibrio cholerae, Bacillus cereus, Listeria monocytogenes, Yersinia enterocolitica, Campylobacter jejuni, Shigella spp., and enteropathogenic Escherichia coli. In one specific aspect of this embodiment, deleterious gastrointestinal levels of Clostridium perfringens are reduced.

In a further embodiment, the present disclosure provides a method for enhancement of gastrointestinal health in an animal in need thereof, the method comprising administration of a composition comprising an effective amount of lactic acid to that animal, as well as the administration of at least one anti-bacterial agent. That anti-bacterial agent can be, for example, an antibiotic, that is administered before, at, or at about the same time, as the composition comprising lactic acid is administered. In one aspect of this embodiment, the antibiotic is combined with the composition comprising lactic acid. In particular aspects of this embodiment, the antibiotic is selected from the group consisting of streptomycin, ampicillin, ciprofloxacin, cephalosporin, clindamycin, neomycin, metronidazole, teicoplanin, teicoplatin, erythromycin, doxycycline, tetracycline, augmentin, cephalexin, penicillin, kanamycin, rifamycin, rifaximin, metronidazole, timidazole, co-trimoxazole, cephamandole, ketoconazole, latamoxef, cefoperazone, amoxicillin, cefmenoxime, furazolidone, vancomycin, and combinations thereof.

In a further embodiment, the present disclosure provides a method for enhancement of gastrointestinal health in an animal in need thereof, the method comprising administration of a composition comprising an effective amount of lactic acid to that animal, as well as the administration of at least one additional anti-diarrhea agent. That anti-diarrhea agent can be administered before, at, or at about the same time, as the composition comprising lactic acid is administered. In one aspect of this embodiment, the anti-diarrhea agent is combined with the composition comprising lactic acid. In specific aspects of this embodiment, the anti-diarrhea agent is selected from the group consisting of loperamide, diphenoxylate, pancrelipase, tincture of opium, peppermint, ginger and combinations thereof.

The present disclosure also provides compositions, including but not limited to pet food compositions that can be administered according to the methods described herein, for enhancing the gastrointestinal health of an animal in need thereof. Such compositions comprise an effective amount of lactic acid and may, in certain embodiments, also comprise an effective amount of one or more agents selected from anti-diarrhea agents, anti-microbial agents such as antibiotics, and fiber. The pet food compositions of the disclosure may, in certain embodiments, be nutritionally-complete pet food compositions.

The present disclosure also provided compositions that are to be administered according to the methods described herein for enhancing the gastrointestinal health of an animal in need thereof that which are prepared in the form of a nutritional supplement, a treat, or a snack. In other aspects of this embodiment, the composition is prepared as a chew or a toy that is at least partially edible by an animal.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

The community of bacteria resident in the gastrointestinal tract comprises both beneficial and deleterious bacterial types or species. Whether a particular member of the gut flora is beneficial, deleterious or inconsequential to the health of the animal in particular circumstances can depend on a number of factors, but for the purposes of the present invention, certain types or species of bacteria can be considered beneficial and others deleterious. Examples of beneficial members of the gut flora include bifidobacteria (species of the genus Bifidobacterium) and lactic acid bacteria, more particularly species of the genus Lactobacillus. Deleterious bacteria include pathogenic bacteria. Examples of deleterious members of the gut flora include Clostridium spp., Desulfovibrio spp. (including without limitation D. desulfuricans, D. intestinalis and D. vulgaris), Helicobacter spp. (including without limitation H. bizzozeronii, H. felis, H. heilmannii, H. pylori and H. salomonis) and pathogenic forms of Escherichia coli.

Gastrointestinal health typically depends on maintenance of an appropriate balance of beneficial and deleterious bacteria. An increase in the population of deleterious bacteria and/or a decrease in the population of beneficial bacteria can be associated with a decline in gastrointestinal health. Conversely, an increase in the population of beneficial bacteria and/or a decrease in the population of deleterious bacteria can be associated with an improvement in gastrointestinal health, for example restoration of health or remission in an animal having a gastrointestinal disorder such as irritable bowel disease (IBD), or prevention of onset of disease in an animal at risk for such a disorder. The phrases “associated with” and “characterized by,” as used in the present context do not necessarily imply a causal relationship, thus the balance of beneficial and deleterious bacteria can be implicated in pathogenesis or can be merely symptomatic of a gastrointestinal disorder.

Bacterial populations in the gut flora can be estimated by any procedure known in the art. For example, stool samples can be cultured using traditional plating methodologies, or illustratively by the fluorescence in situ hybridization (FISH) technique.

As used herein, the term “animal” refers to any animal susceptible to or suffering from poor gastrointestinal health. An animal is “susceptible to” a disease or condition if the animal exhibits symptoms that indicate that the animal is likely to develop the condition or disease or that the animal has been diagnosed as likely to develop the condition or disease using medical criteria, tests, and assays known to those of ordinary skill in the art of animal care. An animal is “suffering from” a disease or condition if the animal exhibits symptoms that are indicative that the animal has developed the condition or disease or if the animal has been diagnosed as afflicted with the disease or condition.

Thus for example a subject animal, i.e., an animal in need of enhancement of gastrointestinal health according to the present method can be one “having or at risk for having IBD.” An animal having IBD is an animal in which any one of a spectrum of inflammatory gastrointestinal diseases and disorders recognized as a form of IBD has been professionally diagnosed or an animal exhibiting symptoms consistent with such diagnosis. Such diseases and disorders include without limitation irritable bowel disease (IBD), ulcerative colitis and Crohn's disease. An animal having chronic IBD but in remission at the time of application of the method is considered herein to be an animal “having IBD.” An animal at risk for having IBD is an animal not having a history of IBD or exhibiting IBD symptoms but having one or more risk factors indicating a susceptibility to development of IBD. Such risk factors can include genetic factors (e.g., a family history of IBD) and physiological factors (e.g., elevated levels of one or more pro-inflammatory biomarkers and/or depressed levels of one or more anti-inflammatory biomarkers).

“About the same time” generally means that the compositions, composition components, anti-diarrhea drugs, anti-microbial agents and food compositions are administered at the same time or within about 72 hours of each other.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

The term “anti-diarrhea drug” means any compound, composition, or drug useful for preventing or treating diarrhea.

As used herein, methods to “treat” an animal suffering from a disease, disorder, or condition are meant to encompass methods to prevent, cure, reverse, attenuate, alleviate, ameliorate, minimize, suppress, or halt the deleterious effects of the disease, disorder, or condition as well.

In one aspect, the present invention provides compositions and methods for enhancing the gastrointestinal health of an animal in need thereof. An animal in need of such enhancement is an animal suffering from or diagnosed as susceptible to a condition related to poor gastrointestinal health. The methods comprise administering a gastrointestinal health enhancing amount of a composition comprising an effective amount of lactic acid to the animal susceptible to or suffering from poor gastrointestinal health. In one non-limiting, illustrative, embodiment, the methods comprise enhancing gastrointestinal health by treating diarrhea. In another, the methods comprise enhancing gastrointestinal health by improving stool quality. In a still further embodiment, the methods comprise enhancing gastrointestinal health by improving nutrient absorption in the gastrointestinal tract. In another embodiment, the methods comprise enhancing gastrointestinal health by treating IBD or reducing the symptoms associated with IBD.

The methods and compositions of the present disclosure are useful for a variety of human and non-human animals susceptible to or suffering from poor gastrointestinal health, including avian, bovine, canine, equine, feline, goat, murine, ovine, and porcine species of animal. In certain embodiments, the animal is a companion animal such as canine or feline, particularly a dog or a cat.

In a specific embodiment, the methods of the present invention comprise administering the composition comprising an effective amount of lactic acid with at least one of a gastrointestinal health improving agent selected from the group consisting of probiotics, prebiotics, synbiotics, and anti-diarrhea drugs. In certain aspects of this embodiment, the agent is administered before, at, or at about the same time as the composition comprising an effective amount of lactic acid is administered. In one aspect of this embodiment, the agent is formulated with the composition comprising an effective amount of lactic acid before administration to the animal in need thereof.

Probiotics useful in the present invention are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics.

Prebiotics are nondigestible food ingredients that beneficially affect host health by selectively stimulating the growth and/or activity of bacteria in the colon. The prebiotic, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit human health by producing compounds to inhibit potential pathogens, by reducing blood ammonia levels, and by producing vitamins and digestive enzymes.

In certain embodiments, the compositions administered comprise both a probiotic and a prebiotic. Where that combination provides a net health benefit is that is synergistic, i.e., where the prebiotic is shown to increase the population and/or function of the probiotic, that combination is referred to as a synbiotic. Examples of synbiotics useful in the present methods include but are not limited to (i) bifidobacteria and fructo-oligosaccharides (FOS), (ii) Lactobacillus rhamnosus GG and inulins, and (iii) bifidobacteria or lactobacilli with FOS or inulins or galactooligosaccharides.

Probiotic bacteria such as Lactobacilli or Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly. Probiotics enhance systemic cellular immune responses and may be useful as a dietary supplement to boost natural immunity in otherwise healthy adults. Probiotics include many types of bacteria but generally are selected from four genera of bacteria: Lactobacilllus acidophilus, Bifidobacteria, Lactococcus, and Pediococcus. The amount of probiotics and prebiotics to be administered to the animal is determined by the skilled artisan based upon the type and nature of the probiotic and prebiotic and the type and nature of the animal, e.g., the age, weight, general health, sex, extent of microbial depletion, presence of harmful bacteria, and diet of the animal. Generally, probiotics are administered to the animal in amounts of from about one to about twenty billion colony forming units (CFUs) per day for the healthy maintenance of intestinal microflora, preferably from about 5 billion to about 10 billion live bacteria per day. Generally, prebiotics are administered in amounts sufficient to positively stimulate the healthy microflora in the gut and cause these “good” bacteria to reproduce. Typical amounts are from about one to about 10 grams per serving or from about 5 percent to about 40 percent of the recommended daily dietary fiber for an animal. The probiotics and prebiotics can be made part of the composition by any suitable means. Generally, the agents are mixed with the composition or applied to the surface of the composition, e.g., by sprinkling When the agents are part of a kit, the agents can be admixed with other materials or in their own package.

The present disclosure is also directed to the described methods of treatment as well as to the compositions, per se, that are administered to an animal in need of treatment.

The present disclosure also provides compositions comprising an effective amount of lactic acid in which the composition is a nutritionally complete pet food composition. A nutritionally complete pet food composition is one that meets the nutritional requirements of the animal to which the composition is to be administered as those requirements are defined, e.g., by AAFCO, as set forth, for example, in the Association of American Feed Control Officials, Inc., Official publication, pp. 159-162 (2011).

In one embodiment, the methods of the disclosure are directed to enhancing the gastrointestinal health of an animal in need thereof comprising feeding that animal a composition comprising an effective amount of lactic acid. In specific aspects of this embodiment, the composition comprises from about 0.01% to about 10% lactic acid. In further aspects of this embodiment, the composition comprises from about 0.02% to about 8% lactic acid, from about 0.05% to about 6.0% lactic acid, from about 0.1% to about 5.0% lactic acid; from about 0.25% to about 4.0% lactic acid, from about 0.25% to about 3.0% lactic acid, from about 0.25% to about 2.0% lactic acid, from about 0.25% to about 2.0% lactic acid, or from about 0.25% to about 1.0% lactic acid.

The methods of this invention contemplate administration of any of a variety of compositions comprising lactic acid to the animal. Contemplated compositions suitable for ingestion by a companion animal include, for example, foods, supplements, treats, snacks and toys (typically chewable and consumable toys or at least partially-consumable or partially-edible toys).

In a further aspect, the present invention provides kits suitable for administering compositions comprising an effective amount of lactic acid to an animal in need thereof according to the presently disclosed methods. The kits comprise in separate containers in a single package or in separate containers in a virtual package, as appropriate for the kit component, at least one composition comprising an effective amount of lactic acid, one of (1) one or more ingredients suitable for consumption by an animal, (2) one or more microbial exopolysaccharides, (3) one or more gastrointestinal tract improving agents selected from the group consisting of probiotics, prebiotics, and synbiotics (4) one or more anti-diarrhea drugs, (5) one or more antibiotics, (6) instructions for how to combine the lactic-acid containing composition(s) and other kit components to enhance gastrointestinal health, particularly in an animal in need thereof, e.g., to produce a composition useful for preventing or treating diarrhea or for improving stool quality, and (7) instructions for how to use compositions comprising an effective amount of lactic acid and other components of the present invention, particularly for the benefit of the animal. When the kit comprises a virtual package, the kit is limited to instructions in a virtual environment in combination with one or more physical kit components. The kit contains the composition comprising an effective amount of lactic acid and other components in amounts sufficient to enhance gastrointestinal health in an animal in need thereof. Typically, the composition comprising an effective amount of lactic acid and the other suitable kit components are admixed just prior to consumption by an animal. In one embodiment, the kit contains a packet containing one or more compositions comprising an effective amount of lactic acid and a container of food for consumption by an animal. The kit may contain additional items such as a device for mixing the composition comprising an effective amount of lactic acid and ingredients or a device for containing the admixture, e.g., a food bowl. In another embodiment, the composition comprising an effective amount of lactic acid is mixed with additional nutritional supplements such as vitamins and minerals that promote good health in an animal.

In another aspect, the present invention provides a means for communicating information about or instructions for one or more of (1) using a composition comprising an effective amount of lactic acid to enhance gastrointestinal health in an animal in need thereof, (2) using compositions comprising an effective amount of lactic acid to improve stool quality in an animal in need thereof, (3) admixing a composition comprising an effective amount of lactic acid with the other components of the present invention, (4) administering a composition comprising an effective amount of lactic acid to an animal, alone or in combination with the other elements of the present invention, and (5) using the kits of the present invention for enhancing gastrointestinal health in an animal in need thereof, e.g., for improving stool quality, comprising a document, digital storage media, optical storage media, audio presentation, or visual display containing the information or instructions. In certain embodiments, the communicating means comprises a document, digital storage media, optical storage media, audio presentation, or visual display containing the information or instructions. Preferably, the communication means is a displayed web site or a brochure, product label, package insert, advertisement, or visual display containing such information or instructions. Useful information includes one or more of (1) methods and techniques for combining and administering a composition comprising an effective amount of lactic acid and/or other components and (2) contact information for animals or their caregivers to use if they have a question about the invention and its use. Useful instructions include amounts for mixing and administration amounts and frequency. The communication means is useful for instructing on the benefits of using the present invention and communicating the approved methods for administering the invention to an animal.

In one embodiment, the composition comprising lactic acid can be fed to the animal as a component of its food intake. The food intake of the animal can meet its ordinary nutritional requirements, which a skilled artisan can determine based upon the animal's species, age, sex, weight, and other factors.

In specific embodiments, the methods of the disclosure are directed to enhancing the gastrointestinal health of an animal in need thereof, comprising feeding that animal a composition comprising from about 0.01% to about 10% lactic acid. In further aspects of this embodiment, the composition comprises from about 0.02% to about 8% lactic acid, from about 0.05% to about 6.0% lactic acid, from about 0.1% to about 5.0% lactic acid; from about 0.25% to about 4.0% lactic acid, from about 0.25% to about 3.0% lactic acid, from about 0.25% to about 2.0% lactic acid, from about 0.25% to about 2.0% lactic acid, or from about 0.25% to about 1.0% lactic acid. In one aspect of such embodiments, the companion animal pet food composition comprises suitable sources of protein, carbohydrate, fat, and fiber.

In a particular aspect of this embodiment, the methods of the disclosure comprise feeding a composition of the disclosure, which composition is a companion animal pet food composition comprising from about 10% to about 50%, from about 15% to about 40%, and from about 20% to about 30% protein. In a specific aspect, the methods of the disclosure comprise feeding a composition of the disclosure, which composition is a companion animal pet food composition comprising about 22% protein.

In a particular aspect of this embodiment, the methods of the disclosure comprise feeding a composition of the disclosure, which composition is a companion animal pet food composition comprising from about 30% to about 70%, from about 35% to about 65%, and from about 40% to about 60% carbohydrate. In a specific aspect, the methods of the disclosure comprise feeding a composition of the disclosure, which composition is a companion animal pet food composition comprising about 50% carbohydrate.

In still another aspect of this embodiment, the methods of the disclosure comprise feeding a composition of the disclosure, which composition is a companion animal pet food composition comprising from about 4% to about 20%, from about 5% to about 16% and from about 6% to about 14% fat. In a specific aspect, the methods of the disclosure comprise feeding a composition of the disclosure, which composition is a companion animal pet food composition comprising about 8% fat.

In another aspect of this embodiment, the methods of the disclosure comprise feeding a composition of the disclosure, which composition is a companion animal pet food composition comprising from about 4% to about 20%, from about 6% to about 18%, from about 8% to about 16% crude fiber, and from about 10% to about 14% crude fiber. In a specific aspect, the methods of the disclosure comprise feeding a composition of the disclosure, which composition is a companion animal pet food composition comprising about 12% crude fiber.

In another particular aspect of this embodiment, the methods of the disclosure comprise feeding a composition of the disclosure, which composition is a companion animal pet food composition comprising about 22% protein, about 50% carbohydrate, about 8% fat, about 12% crude fiber and from about 0.01% to about 10% lactic acid.

The pet food compositions disclosed above are particularly suitable for feeding to canines in need of enhancing their gastrointestinal health. In other embodiments, pet food compositions particularly suitable for use in the disclosed methods of treatment of feline companion animals generally have some higher levels of protein but lower levels of carbohydrate and fiber than described above.

Therefore, in another embodiment, the methods of the disclosure are directed to enhancing the gastrointestinal health of a feline animal in need thereof, comprising feeding that feline a composition comprising from about 0.01% to about 10% lactic acid. In further aspects of this embodiment, the composition comprises from about 0.02% to about 8% lactic acid, from about 0.05% to about 6.0% lactic acid, from about 0.1% to about 5.0% lactic acid; from about 0.25% to about 4.0% lactic acid, from about 0.25% to about 3.0% lactic acid, from about 0.25% to about 2.0% lactic acid, from about 0.25% to about 2.0% lactic acid, or from about 0.25% to about 1.0% lactic acid. In one aspect of such embodiments, the companion animal pet food composition comprises suitable sources of protein, carbohydrate, fat, and fiber.

In a particular aspect of this embodiment, the methods of the disclosure comprise feeding a composition of the disclosure, which composition is a companion animal pet food composition comprising from about 20% to about 45%, from about 25% to about 40%, and from about 30% to about 35% protein. In a specific aspect, the methods of the disclosure comprise feeding a composition of the disclosure, which composition is a companion animal pet food composition comprising about 33% protein.

In a particular aspect of this embodiment, the methods of the disclosure comprise feeding a composition of the disclosure, which composition is a companion animal pet food composition comprising from about 15% to about 45%, from about 20% to about 40%, and from about 25% to about 35% carbohydrate. In a specific aspect, the methods of the disclosure comprise feeding a composition of the disclosure, which composition is a companion animal pet food composition comprising about 33% carbohydrate.

In still another aspect of this embodiment, the methods of the disclosure comprise feeding a composition of the disclosure, which composition is a companion animal pet food composition comprising from about 5% to about 35%, from about 10% to about 30%, and from about 15% to about 25% fat. In a specific aspect, the methods of the disclosure comprise feeding a composition of the disclosure, which composition is a companion animal pet food composition comprising about 19% fat.

In a another aspect of this embodiment, the methods of the disclosure comprise feeding a composition of the disclosure, which composition is a companion animal pet food composition comprising from about 1% to about 15%, from about 2% to about 12% and from about 3% to about 10% crude fiber. In a specific aspect, the methods of the disclosure comprise feeding a composition of the disclosure, which composition is a companion animal pet food composition comprising about 3% crude fiber.

In a particular aspect of this embodiment, the methods of the disclosure comprise feeding a composition of the disclosure, which composition is a companion animal pet food composition comprising about 33% protein, about 33% carbohydrate, about 19% fat, about 3% crude fiber and from about 0.01% to about 10% lactic acid.

In a further aspect, the method of the present disclosure comprises administering a composition comprising an effective amount of lactic acid, wherein the composition further comprises at least one microbial exopolysaccharide. Microbial exopolysaccharides useful in the compositions and methods described herein include microbial exopolysaccharides capable of preventing or treating diarrhea. Examples of suitable microbial exopolysaccharides for use in the present invention generally include those microbial exopolysaccharides synthesized from bacteria selected from the group consisting of Sphingomonas paucimobilis, Agrobacterium biovar, Xanthomonas campestris, Alcaligenes species, Aureobasidium pullulans, Acetobacter xylinum, Sclerotium rolfsii, Schizophyllum commune, Saccharomyces cerevisiae and Sclerotium glucanicum. In some embodiments, the microbial exopolysaccharide is selected from the group consisting of rhamsan, curdlan, xanthan gum, scleroglucan, PS-10 gum, PS-21 gum, PS-53 gum, polysaccharides from Alcaligenes species, PS-7 gum, gellan gum, curdlan, bacterial alginate, dextran, pullulan, baker's yeast glycan, bacterial cellulose, 6-deoxy-hexose-containing polysaccharides, and combinations thereof.

In a specific embodiment, the microbial exopolysaccharide comprises a gellan gum. Gellan gum is a linear polysaccharide made from fermentation by Sphingomonas paucimobilis (elodea) (ATCC31461). Industrial preparation of the gum can be carried out by inoculating Sphingomonas paucimobilis into a fermentation broth containing glucose, glucuronic acid and rhamnose to form a tetrasaccharide repeating unit in a ratio of 2:1:1. In its native form, gellan gum is highly acylated with 1.5 acyl group, acetyl and glycerate, per repeating unit. Modifications of the acyl groups both in number and type can be made as long as the basic anti diarrhea activity of the gellan gum is not significantly diminished. These different forms can be obtained from CP Kelco under different tradenames including Gelrite®, K9A50 and other Kelco gellan gums including but not limited to, Kelcogel LT®, Kelcogel F, and Kelcogel LT100®. As used throughout the specifications “gellan” refers to the natural gum or acyl modified gum as long as the anti-diarrhea function is maintained.

Generally, the microbial exopolysaccharide is administered in amounts of from about 0.05 to about 2 g/kg body weight per day.

In another illustrative example, a nutritionally-complete companion animal pet food composition for canine companion animals may further comprise, inter alia, vitamins, minerals and other additives. In one aspect of this embodiment, this pet food composition comprises from about 0.01% to about 10% lactic acid. In further aspects of this embodiment, the composition comprises from about 0.02% to about 8% lactic acid, from about 0.05% to about 6.0% lactic acid, from about 0.1% to about 5.0% lactic acid; from about 0.25% to about 4.0% lactic acid, from about 0.25% to about 3.0% lactic acid, from about 0.25% to about 2.0% lactic acid, from about 0.25% to about 2.0% lactic acid, or from about 0.25% to about 1.0% lactic acid, and may further comprise whole grain corn, soybean mill run, chicken by-product meal, powdered cellulose, corn gluten meal, soybean meal, chicken liver flavor, soybean oil, flaxseed, caramel color, iodized salt, L-lysine, choline chloride, potassium chloride, vitamin E supplement, vitamins (L-ascorbyl-2-polyphosphate (source of vitamin C), vitamin E supplement, niacin, thiamine mononitrate, vitamin A supplement, calcium pantothenate, biotin, vitamin B₁₂ supplement, pyridoxine hydrochloride, riboflavin, folic acid, vitamin D₃ supplement), minerals (ferrous sulfate, zinc oxide, copper sulfate, manganous oxide, calcium iodate, sodium selenite), taurine, L-carnitine, preserved with mixed tocopherols and citric acid, phosphoric acid, beta-carotene, and rosemary extract.

In another illustrative example, a nutritionally-complete companion animal pet food composition for feline companion animals may further comprise, inter alia, vitamins, minerals and other additives. In one aspect of this embodiment, this pet food composition comprises an effective amount of lactic acid and may further comprise brewers rice, corn gluten meal, chicken by-product meal, powdered cellulose, whole grain corn, chicken liver flavor, animal fat (preserved with mixed tocopherols and citric acid), soybean mill run, calcium sulfate, choline chloride, potassium chloride, iodized salt, taurine, DL-methionine, vitamin E supplement, vitamins (L-ascorbyl-2-polyphosphate (source of vitamin C), vitamin E supplement, niacin, thiamine mononitrate, vitamin A supplement, calcium pantothenate, riboflavin, biotin, vitamin B₁₂ supplement, pyridoxine hydrochloride, folic acid, vitamin D₃ Supplement), minerals (ferrous sulfate, zinc oxide, copper sulfate, manganous oxide, calcium iodate, sodium selenite), L-carnitine, preserved with mixed tocopherols and citric acid, phosphoric acid, beta-carotene, and rosemary extract.

In another embodiment, the methods of the disclosure comprise feeding a composition of the disclosure, which composition is a companion animal pet food composition as described above, and further comprising an antioxidant. In particular, such compositions comprise an oral health-promoting effective total amount of at least one antioxidant. In certain aspects, the antioxidant is selected from the group consisting of vitamin C, vitamin E, vitamin A, lipoic acid, astaxanthin, beta-carotene, L-carnitine, coenzyme Q10, glutathione, lycopene, lutein, N-acetylcysteine, soy isoflavones, S-adenosylmethionine, taurine, tocotrienols, spinach, tomato, citrus fruit, grape, carrot, broccoli, green tea, ginkgo biloba, corn gluten meal, rice bran, algae, curcumin, marine oil, fruits, vegetables, yeast, carotenoids, flavonoids, polyphenols, and mixtures thereof.

In certain embodiments, the antioxidant-comprising composition can comprise vitamin E, vitamin C, or both vitamin E and vitamin C. In one aspect of this embodiment, the vitamin E content of a composition can be at least about 100 ppm, illustratively about 100 to about 5000 ppm, about 250 to about 2500 ppm, or about 500 to about 1500 ppm. In another aspect of this embodiment, the vitamin C content of a composition can be at least about 10 ppm, illustratively about 10 ppm to about 10,000 ppm, or about 20 to about 2000 ppm, or about 25 to about 500 ppm.

In certain embodiment, compositions of the present invention are formulated as companion animal pet food compositions. In one aspect of this embodiment, the pet food compositions are nutritionally complete pet food compositions.

In particular embodiments, the pet food compositions, and particularly the nutritionally-complete pet food compositions of the present disclosure, can be prepared in a dry form using conventional processes. In one contemplated embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated with one or more topical coatings which can include, for example, flavors, fats, oils, powders, and the like. Kibble also can be made from the dough using a baking process, rather than extrusion, wherein the dough is placed into a mold before dry-heat processing. Kibble also can be made from a food matrix undergoing pelletization. It particular aspects of this embodiment, lactic acid is incorporated into the food composition by adding it to the above-described mixtures before extrusion or by coating the extruded kibble or pellets with, for example, lactic acid as an ingredient of a topical coating.

Treats of the present invention can be prepared by, for example, an extrusion or baking process similar to those described above for dry food. Other processes also can be used to either apply a coating comprising lactic acid on the exterior of existing treat forms, or inject lactic acid into an existing treat form.

In another embodiment, the composition is a food supplement comprising lactic acid. Supplements include, for example, a feed or pet food used with another feed or pet food to improve the nutritive balance or performance of the total. Contemplated supplements include compositions that are fed undiluted as a supplement to other feeds or pet foods, offered free choice with other parts of an animal's ration that are separately available, or diluted and mixed with an animal's regular feed or pet food to produce a complete feed or pet food. AAFCO, for example, provides a discussion relating to supplements in the Association of American Feed Control Officials, Inc., Official Publication (2011). Supplements can be in various forms including, for example, powders, liquids, syrups, pills, encapsulated compositions, etc.

In another embodiment, the composition is a treat comprising lactic acid. Treats include, for example, compositions that are given to an animal to entice the animal to eat during a non-meal time. Contemplated treats for canines include, for example, dog biscuits in the shape of dog bones. Treats can be nutritional, wherein the composition comprises one or more nutrients, and can, for example, have a composition as described above for food. Non-nutritional treats encompass any other treats that are non-toxic. A lactic acid containing composition can, for example, be coated onto the treat, incorporated into the treat, or both.

In another embodiment, the composition is a toy comprising lactic acid. Toys include, for example, chewable toys. Contemplated toys for dogs include, for example, artificial bones. The lactic acid or lactic acid-containing composition, for example, can be present in a coating on the surface of the toy or on the surface of a component of the toy, or can be incorporated partially or fully throughout the toy, or both. In a contemplated embodiment, lactic acid is orally accessible by the intended user.

Illustrative toys suitable for modification in accordance with the invention include those described in U.S. Pat. No. 5,339,771 and references disclosed therein, and in U.S. Pat. No. 5,419,283 and references disclosed therein, which are incorporated herein by reference.

It should be recognized that this disclosure contemplates both partially consumable toys (e.g., toys comprising plastic components) and fully consumable toys (e.g., rawhides and various artificial bones). It should be further recognized that this invention contemplates toys for both human and non-human use, particularly for companion, farm, and zoo animal use, and particularly for dog or cat use.

The terms “treat” and “toy” can be considered interchangeable for the purposes of this specification. However, in general a treat is fully edible and a toy in accordance with the invention has an edible coating.

In preparing a composition of the present invention, lactic acid can, for example, be incorporated into the composition during formulation processing, such as during and after mixing of other components of the composition. Distribution of these components into the composition can be accomplished by any conventional method including standard mixing procedures.

In certain embodiments, the compositions of the present disclosure, whether a companion animal pet food composition, dietary supplement, treat or toy, may further comprise at least one a commercial liquid palatant enhancer or other flavor composition to create a novel flavor palatant which can then be included within or topically applied to the composition. Suitable commercial liquid palatant enhancers for use with compositions of the present disclosure can include any known or commercially available liquid palatant enhancers commercially available from pet food palatant enhancer or other flavor suppliers known to those of skill in the art. In one illustrative aspect, the kibble, treat, or toy can be coated with a composition comprising lactic acid and a carrier, wherein the carrier comprises protease-treated minced animal by-product, amino acids, one or more reducing sugars and thiamin.

In certain embodiments, the methods of the disclosure may further comprise administration of an antibiotic to the animal in need of gastrointestinal health enhancement. The antibiotic may be administered before, at, or at about the same time as the composition comprising an effective amount of lactic acid is administered to that animal in need of gastrointestinal health enhancement. In particular aspects of this embodiment, the antibiotic administered may be one or may be a mixture of one or more of streptomycin, ampicillin, ciprofloxacin, cephalosporin, clindamycin, neomycin, metronidazole, teicoplanin, teicoplatin, erythromycin, doxycycline, tetracycline, augmentin, cephalexin, penicillin, kanamycin, rifamycin, rifaximin, metronidazole, timidazole, co-trimoxazole, cephamandole, ketoconazole, latamoxef, cefoperazone, amoxicillin, cefmenoxime, furazolidone, and vancomycin.

In certain embodiments, the method of enhancing gastrointestinal health in an animal in need thereof is achieved by reducing the level of at least one genus or species of deleterious bacteria in the gastrointestinal tract. Examples of genera of deleterious bacteria include but are not limited to Enterococcus, Desulfovibrio, and Helicobacter. In a specific embodiment, practice of the disclosed method of enhancing gastrointestinal health in an animal will result in a decrease in the total load or bacterial count of organisms classified as falling within the genus Enterococcus.

In another embodiment, the method of enhancing gastrointestinal health in an animal in need thereof is achieved by reducing the gastrointestinal level of one or more species of deleterious bacteria selected from among Salmonella typhimurium, Staphylococcus aureus, Clostridium perfringens, Clostridium difficile, Vibrio parahaemolyticus, Vibrio cholerae, Bacillus cereus, Listeria monocytogenes, Yersinia enterocolitica, Campylobacter jejuni, Shigella spp., and enteropathogenic Escherichia coli. In a specific embodiment, practice of the disclosed method of enhancing gastrointestinal health in an animal will result in a decrease in the total load or bacterial count of organisms classified as Clostridium perfringens.

### EXAMPLES

### Example 1

### Effect of Dietary Lactic Acid on Gastrointestinal Microbial Load

The effect of administration of dietary lactic acid on the level of gastrointestinal levels of microorganisms was investigated in cats. A group of ten cats was divided into two sub-groups of five cats each. One sub-group was fed a control diet for 7 days and the other sub-group a test diet containing lactic acid. After the seven day period, the cats were switched to the test diet or control diet, respectively, and fed for a second seven-day period. The ingredients in the control (labeled Feline Control Diet) and the test diet (labeled Feline Test Diet) are as analyzed in Table 1.

TABLE 1

| | Feline Control | Feline Test |
|---|---|---|
| | Diet | Diet |
| Ingredient | Amount (%) | Amount (%) |
| Moisture | 6.6 | 7.5 |
| Protein | 33.1 | 31.5 |
| Fat | 18.9 | 19.4 |
| Crude Fiber | 3.2 | 2.7 |
| Ash | 5.0 | 5.2 |
| NFE | 33.3 | 33.8 |

Stool samples were collected for all groups and counts determined for each of the indicated organism included in Table 2.

TABLE 2
Overall Mean (log cfu/g) In Cats Fed Control And Test Diets

| | Feline Control | Feline Test |
|---|---|---|
| Microorganism | Diet | Diet |
| Aerobic | 6.9 | 6.4 |
| Anaerobic | 7 | 6.5 |
| Bacillus | 1.9 | 1.7 |
| Coliforms | 5.5 | 5.2 |
| Enterococci | 4.8 | 3.9 |
| Lactobacillus | 4.3 | 4.1 |
| Mold Count | 3.7 | 3.6 |

The data of Table 2 indicate that feeding cats a pet food composition comprising lactic acid can result in a decrease in the level of all of the microorganisms tested.

### Example 2

### Effect of Dietary Lactic Acid on Gastrointestinal Load of Bacterial Species

The effect of administration of dietary lactic acid on the gastrointestinal levels of specific bacterial strains was investigated in dogs. The dogs were fed either a control diet or a test diet comprising lactic acid and fiber for at least 7 days. Two control diets were used in the study. The first control diet (labeled Canine Control Diet #1) was formulated to contain the ingredients as in Table 3.

TABLE 3

| | Canine Control |
|---|---|
| | Diet #1 |
| Ingredient | Amount (%) |
| Moisture | 8.0 |
| Protein | 23.0 |
| Fat | 10.1 |
| Crude Fiber | 2.3 |
| Ash | 4.4 |

The ingredients in the second control diet (labeled Canine Control Diet #2) and the test diet (labeled Canine Test Diet) are as analyzed in Table 4.

TABLE 4

| | Canine Control | Canine Test |
|---|---|---|
| | Diet #2 | Diet |
| Ingredient | Amount (%) | Amount (%) |
| Moisture | 8.6 | 8.2 |
| Protein | 22.0 | 22.1 |
| Fat | 8.1 | 8.2 |
| Crude Fiber | 11.0 | 11.8 |
| Ash | 4.5 | 4.3 |

Stools were then collected and defined bacterial species identified and quantified using gene hybridization technology. The data obtained are presented in Table 5.

TABLE 6
Overall Mean (log cfu/g) in Dogs Fed Control And Test Diets

| | Canine Control | Canine Control | Canine Test |
|---|---|---|---|
| Microorganism | Diet #1 | Diet #2 | Diet |
| Clostridium | 9.8 | 9.1 | 9.4 |
| perfringens | | | |
| Escherichia coli | 10.8 | 10.5 | 10.2 |
| Lactobacilli | 11.1 | 10.3 | 9.8 |
| Bifidobacteria | 9.1 | 8.9 | 8.6 |

The data of Table 6 indicate that feeding dogs a pet food composition comprising lactic acid and fiber can result in a decrease in the level of each of the bacterial species identified.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

What is claimed is:

1. A method for enhancing the gastrointestinal health of an animal in need thereof, the method comprising administering to said animal a composition comprising an effective amount of lactic acid.

2. The method of claim 1, wherein the amount of lactic acid is about 0.01% to about 10%.

3. The method of claim 1 or claim 2, wherein said animal is diagnosed as suffering from or is diagnosed as susceptible to a condition related to poor gastrointestinal health.

4. The method of claim 3, wherein the condition comprises diarrhea.

5. The method of claim 3, wherein the condition comprises irritable bowel disorder.

6. The method of claim 3, wherein the condition comprises nutrient malabsorption.

7. The method of any preceding claim, wherein the animal is a canine or feline.

8. The method of any preceding claim, wherein the composition further comprises an effective amount of an ingredient selected from the group consisting of probiotics, prebiotics, synbiotics, microbial exopolysaccharides, and combinations thereof.

9. The method of any preceding claim, wherein the composition is a nutritionally-complete pet food composition.

10. The method of claim 9, wherein the composition is a dry pet food composition.

11. The method of claim 10, wherein the dry pet food composition comprises a lactic acid containing coating.

12. The method of claim 9, wherein the composition is a wet pet food composition.

13. The method of any preceding claim, wherein the composition is a food supplement.

14. The method of any preceding claim, wherein the composition is in the form of a treat, a snack, or an at least partially edible chew, or toy.

15. The method of any preceding claim, wherein the enhancement comprises a decrease in the gastrointestinal level of at least one genus or species of deleterious bacteria.

16. The method of claim 14, wherein the deleterious bacterial genus is selected from among Enterococcus, Desulfovibrio, and Helicobacter.

17. The method of claim 15, wherein the deleterious bacterial genus is Enterococcus.

18. The method of claim 14, wherein the deleterious bacterial species is selected from the group consisting of Salmonella typhimurium, Staphylococcus aureus, Clostridium perfringens, Clostridium difficile, Vibrio parahaemolyticus, Vibrio cholerae, Bacillus cereus, Listeria monocytogenes, Yersinia enterocolitica, Campylobacter jejuni, Shigella spp., and enteropathogenic Escherichia coli.

19. The method of claim 17, wherein the deleterious bacterial species is Clostridium perfringens.

20. The method of any preceding claim, further comprising administration of an antibiotic, before, at, or at about the same time as administration of the composition comprising lactic acid.

21. The method of claim 19, wherein the antibiotic is selected from the group consisting of streptomycin, ampicillin, ciprofloxacin, cephalosporin, clindamycin, neomycin, metronidazole, teicoplanin, teicoplatin, erythromycin, doxycycline, tetracycline, augmentin, cephalexin, penicillin, kanamycin, rifamycin, rifaximin, metronidazole, timidazole, co-trimoxazole, cephamandole, ketoconazole, latamoxef, cefoperazone, amoxicillin, cefmenoxime, furazolidone, vancomycin, and combinations thereof.

22. The method of any preceding claim, further comprising administration of an anti-diarrhea drug, before, at, or at about the same time as administration of the composition comprising lactic acid.

23. The method of claim 21, wherein the anti-diarrhea drug is selected from the group consisting of loperamide, diphenoxylate, pancrelipase, tincture of opium, peppermint, ginger and combinations thereof.

24. A composition useful for enhancing the gastrointestinal health of an animal comprising a gastrointestinal health enhancing amount of lactic acid, wherein the composition is a nutritionally-complete companion animal pet food composition comprising at least one of a gastrointestinal health improving agent selected from the group consisting of probiotics, prebiotics, syntbiotics, anti-diarrhea drugs, antibiotics, microbial exopolysaccharides, and combinations thereof.
